(19) United States
(12) Patent Application Publication
(10) Pub. No.: US 2024/0016657 A1
(43) Pub. Date: Jan. 18, 2024

(54) MYBROWS APPARATUS

(71) Applicant: SAVE THE BROWS, LLC, San Fernando, CA (US)

(72) Inventor: Jeremy Needleman, San Fernando, CA (US)

(21) Appl. No.: 18/221,924

(22) Filed: Jul. 14, 2023

Related U.S. Application Data
(60) Provisional application No. 63/389,286, filed on Jul. 14, 2022.

(51) Int. Cl. A61F 7/10

(52) U.S. Cl. CPC A61F 7/10; A61F 2007/108; A61F 2007/0004

(57) ABSTRACT

A brow saving device, including a first end portion, a second end portion; a middle section adjacent to the first end portion and the second end portion; a hook at an end of the first end portion; a loop at an end of the first end portion configured to connect the loop to the hook; and a pocket to an inside portion of the middle section, the pocket configured to hold two or more ice packets.

### RELATED APPLICATIONS

This application claims priority to U.S. provisional patent application Ser. No. 63/389,286, filed Jul. 14, 2022, entitled “MyBrows Apparatus,” the disclosure of which is incorporated by reference herein in its entirety.

### TECHNICAL FIELD

This disclosure relates to an apparatus for use in preserving an individual's eyebrow during medical treatments and specifically during chemotherapy treatments.

### BACKGROUND

Chemotherapy and other cancer treatments are very damaging to a patient's body. One of the common side effects of chemotherapy is the loss of hair. The loss of a patient's hair includes a patient losing their eyebrows. In some cases, patients spend a lot of time taking care of their eyebrows. Thus, the loss of a patient's eyebrows can be emotionally damaging in addition to the physical damage that the patient's body is experiencing with regard to chemotherapy or other cancer treatments. Accordingly, a need exists to develop an apparatus or device to specifically focus on saving a patient's eyebrows.

### BRIEF DESCRIPTION OF DRAWINGS

FIG. 1A illustrates a front or outside view of a brow saving device according to some embodiments;

FIG. 1B a back or inside view of a brow saving device according to some embodiments; and

FIG. 2 is a view of a brow saving device installed or placed on an individual's head according to some embodiments.

### DETAILED DESCRIPTION

A better understanding of the features, advantages and principles of the present disclosure will be obtained by reference to the following detailed description that sets forth illustrative embodiments, and the accompanying drawings. Although the detailed description includes many specific embodiments, these are provided by way of example only and should not be construed as limiting the scope of the inventions disclosed herein.

Described herein is a brow saving device utilized to protect a patient's brow during chemotherapy or other damaging cancer treatments. The brow saving device comprises a headband that wraps around a patient's head and specifically covers both of the patient's eyebrows with pockets that allow for the easy swapping or replacement of ice packs.

The brow saving device comprises a headband that wraps around a patient's head and specifically covers both of the patient's eyebrows with pockets that allow for the easy swapping or replacement of ice packs. Although the headband may also cover a portion of the patient's hair (depending on the patient's hair style), the focus of the brow saving device is to cover the patient's eyebrows. The brow saving device may include a pouch or pocket where an ice pack can be inserted. When the brow saving device is worn by the user or patient, the ice pack that is inserted or positioned in the pocket and may be pressed against the patient's eyebrows. The pressing of a cold surface against the user or patient's eyebrows has been found to assist in keeping the user or patient's eyebrows from falling out and/or thinning. It is important to limit the size so to not apply too much cold or ice to user's or patient's forehead. The brow saving device may be made of a comfortable fabric because it will be touching a patient's face, forehead and back of a patient's headband. In some embodiments, a body of the brow saving device may be made of a nylon or spandex material. In some embodiments, the body of the brow saving device may include a ½ inch finished binding around a circumference and/or perimeter of the brow saving device.

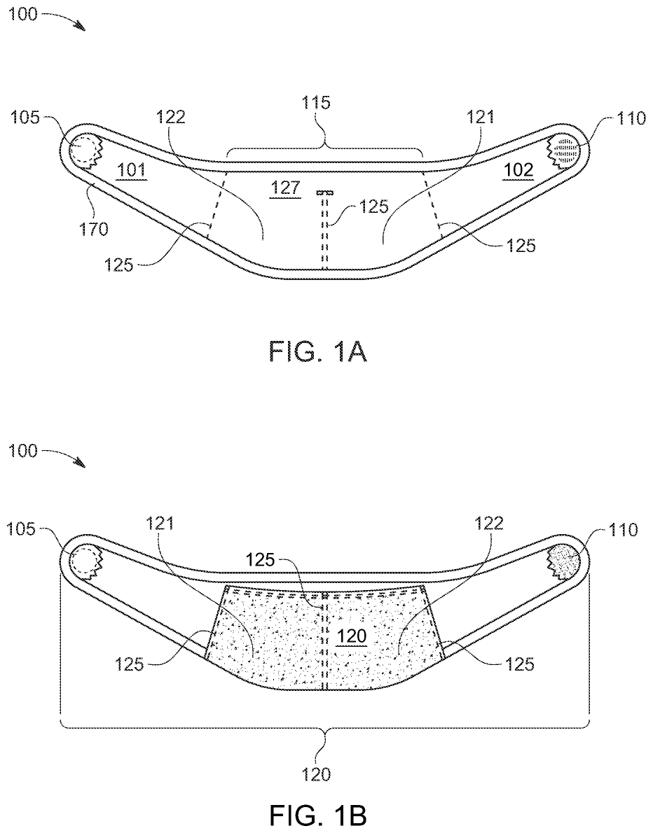

FIG. 1A illustrates a front or outside view of a brow saving device according to some embodiments. FIG. 1B a back or inside view of a brow saving device according to some embodiments. In some embodiments, the brow saving device 100 may be 23½ inches in length. In some embodiments, the brow saving device 100 may range in length from 15 inches to 40 inches. In some embodiments, the length of the brow saving device 100 may be determined based on a size of the patient's head.

The brow saving device 100 may wrap around a patient's head. In some embodiments, the brow saving device 100 may wrap around just above eye level of an individual. In some embodiments, the brow saving device 100 may wrap around a patient's head and may touch a patient's or user's eyebrows.

In some embodiments, the brow saving device 100 may include a Velcro fastening system or fastener. In some embodiments, the brow saving device 100 may include a rough/hook Velcro fastener 105 on one end and a soft/loop Velcro fastener on a second and opposite end 110. Other fastening apparatuses and/or devices may also be utilized to connect one end 105 of the brow saving device 100 to the opposite end 110 of the brow saving device 100 including, but not limited to a hook and latch connector, a snap connector assembly, or another adhesive assembly. In some embodiments, the fastening device of the brow saving device 100 may allow an overall length of a brow saving device to be adjustable.

In some embodiments, as illustrated in FIGS. 1A and 1B, a middle section 115 may be wider than end portions 101 and 102 of the brow saving device 110. This allows less of a user or patient's hair to be touched and/or contacted by the brow saving device 100 and for the focus to be on the patient's brow. In other embodiments, the middle section 115 may be the same width as the end portions 101 and 102 of the brow saving device 100. In some embodiments, the middle section 115 may include a first middle section 127 and a second middle section 128. In some embodiments, a middle section 115 may have a length of 10 inches. In some embodiments, a length of a middle section 115 may have a length ranging from 5 to 18 inches.

In some embodiments, an inside part of the middle section 115, as shown in FIG. 1B of the brow saving device 100 may include a sheer stretch lace pocket 120, although other flexible and/or comfortable materials may be utilized. Any material that allows cold or heat to pass through the material may be utilized in sheer stretch lace pocket 120. In some embodiments, the pocket 120 may be ten inches in length at a bottom portion and/or 9 inches in length at a top portion. In other embodiments, the top portion of the pocket 120 may be the same length as the bottom portion. In some embodiments, the pocket's 120 may range in length from 4 inches to 18 inches. In some embodiments, the length of the pocket 120 may be determined based on a size of a patient's head and/or a space between the user's or patient's or eyebrows. The important design factors are that the pocket 120 needs to be placed against the patient's eyebrows and should not move too much.

In some embodiments, the bottom portion of the pocket 120 may be a little longer than the top portion of the pocket 110 (as is illustrated in FIG. 1B). In some embodiments, the pocket 120 may have a left portion 121 and a right portion 122, which may be approximately equal in area. In some embodiments, in FIG. 1B, two ice packets (not shown) may be inserted and/or placed in the left portion 121 and the right portion 122 of the pocket 120. In some embodiments, the left portion 121 and the right portion 122 of the pocket 120 may be sealed at an end in order to keep the ice packets in the left portion 121 and the right portion 122 in place. In some embodiments, the left portion 121 and the right portion 122 of the pocket may be sealed by a zipper, a zip-lock type connection, a snap connector or other connection assemblies. In some embodiments, one side of the left portion 121 and the right portion 122 may be stitched closed so that only one side of the left portion 121 and the right portion 122 is open for insertion of ice packets. In some embodiments, the left portion 121 and the right portion 122 of the pocket 120 may be formed due to stitching 125 that connects the pocket 120 to the body of the brow saving device 100. In some embodiments, there may be three vertical stiches 125 in order to create the left portion 121 and the right portion 122 of the pocket.

Because the pocket 120 is made of a sheer and/or nylon material, the coldness of the ice packets may be felt against patient or user's eyebrows and may protect the patient or user's eyebrow from falling out and/or thinning. In some embodiments, the brow saving device 100 may only have one pocket 120 (not two as is shown in FIGS. 1A and 1B) and there may not be any stitching in the middle of the pocket 120. In some embodiments, the pocket 120 may have a length of 10 inches. In some embodiments, the right portion 121 of the pocket may have a length of 5 inches and the left portion 122 of the pocket may have a length of 5 inches. In some embodiments, the length of the left portion 122 and the right portion 121 of the pocket 120 may have a length ranging from 2 to 8 inches. In some embodiments, the pockets may have a height ranging from 2 inches to 5 inches.

In some embodiments, most of the portions of the brow saving device 100 may be made of a single piece of material, except for the Velcro connectors 105 and 110, and the pocket 120 (the left portion 122 and the right portion 121).

In some embodiments, the brow saving device 100 may be finished by a stitching or a binding 170. In some embodiments, the stitching or binding 170 may be a nylon and/or spandex material). In some embodiments, the stitching or binding may be ½″ in width. In some embodiments, the stitching or binding 170 may be positioned or installed around a circumference of the brow saving device 170. In some embodiments, the brow saving device 100 may be a nylon, a spandex, a cloth, a plastic and/or any combination thereof material. The brow saving device 100 may be utilized for 30 minutes (e.g., pressed against a patient's eyebrows for 30 minutes). Alternatively, the brow saving device 100 may be utilized for 1 hour or two hours at a time. Alternatively, the brow saving device 100 may be utilized for 15 minutes to 30 minutes at a time. The brow saving device 100 may be placed against the user's brow a number of times during a 24-hour period, for example, from 4 to 14 times.

The brow saving device may also have a width of 3 inches. The brow saving device may also have a width ranging from 1.5 inches to 6 inches.

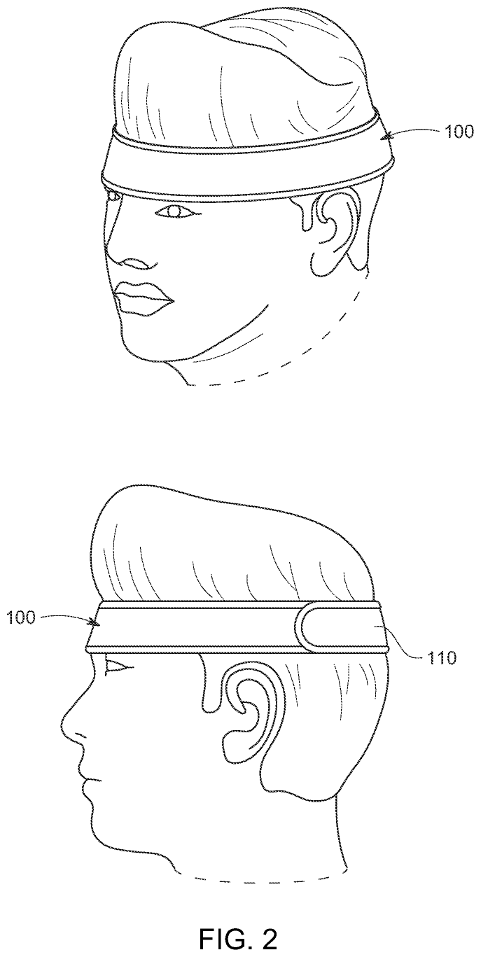

FIG. 2 is a view of a brow saving device installed or placed on an individual's head according to some embodiments. In some embodiments, the brow saving device 100 may be wrapped around or positioned around a patient's or user's head at an eyebrow level. In some embodiments, by positioning the brow saving device 100 around the user's or patient's head at an eyebrow level, a large portion of the patient's head (e.g., the top of the head) may be exposed. In addition, the ice packets are resident in the pocket 120 (e.g., the left portion 122 or the right portion 121) and thus no ice or cold temperature is being placed against a user's temple or other areas of a skull.

In some embodiments, a brow saving device may include a first end portion; a second end portion; a middle section adjacent to the first end portion and the second end portion; a hook at an end of the first end portion; a loop at an end of the first end portion configured to connect the loop to the hook; and a pocket to an inside portion of the middle section, the pocket configured to hold two or more ice packets. In some embodiments, the first end portion, the second end portion, and the middle section may be made of a single piece of material. In some embodiments, the pocket may include a first portion and a second portion, the first portion may be configured to a first one of the two or more ice packets and the second portion may be configured to hold a second one of the two more ice packets. In some embodiments, an end of the first portion may be configured to allow insertion of the first one of the two or more ice packets and an end of the second portion may be configured to allow insertion of the second one of the two or more ice packets. In some embodiments, a top of the first portion of the pocket may be sewn shut and a top of the second portion may be sewn shut. In some embodiments, a middle stitching may separate the first portion of the pocket from the second portion of the pocket. In some embodiments, a circumference of the brow saving device may be finished with a nylon or spandex binding.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

As used herein, the term “or” is used inclusively to refer items in the alternative and in combination.

As used herein, characters such as numerals refer to like elements.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. A brow saving device, comprising:
a first end portion;
a second end portion;
a middle section adjacent to the first end portion and the second end portion;
a hook at an end of the first end portion;
a loop at an end of the first end portion configured to connect the loop to the hook; and
a pocket to an inside portion of the middle section, the pocket configured to hold two or more ice packets.

2. The brow saving device of claim 1, wherein the first end portion, the second end portion, and the middle section are made of a single piece of material.

3. The brow saving device of claim 1, wherein the pocket includes a first portion and a second portion, the first portion configured to a first one of the two or more ice packets and the second portion configured to hold a second one of the two more ice packets.

4. The brow saving device of claim 3, wherein an end of the first portion is configured to allow insertion of the first one of the two or more ice packets and an end of the second portion is configured to allow insertion of the second one of the two or more ice packets.

5. The brow saving device of claim 4, wherein a top of the first portion of the pocket is sewn shut and a top of the second portion is sewn shut.

6. The brow saving device of claim 5, wherein a middle stitching separates the first portion of the pocket from the second portion of the pocket.

7. The brow saving device of claim 1, wherein a circumference of the brow saving device is finished with a nylon or spandex binding.
